(19) United States
(12) Patent Application Publication
(10) Pub. No.: US 2004/0033543 A1
(43) Pub. Date: Feb. 19, 2004

(54) Treatment of renal carcinoma using antibodies against the EGFr

(76) Inventors: Gisela Schwab, Hayward, CA (US); Xiao-Dong Yang, Palo Alto, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/441,648

(22) Filed: May 19, 2003

Related U.S. Application Data
(60) Provisional application No. 60/382,152, filed on May 20, 2002.

(51) Int. Cl. G01N 33/574; A61K 39/395

(52) U.S. Cl. 435/7.23; 424/143.1

(57) ABSTRACT

Methods of treating renal carcinoma are described using fully human monoclonal antibodies ABX-EGF against the epidermal growth factor receptor (EGFr) and antigen binding fragments thereof. Methods of using these renal carcinoma treatments specifically as a monotherapy are also described. In addition, a kit and an article of manufacture for the treatment of renal carcinoma treatment are provided.

### REFERENCE TO RELATED APPLICATION

The present application claims the priority benefit under 35 U.S.C. §119(e) of Provisional Application No. 60/382,152, filed on May 20, 2002.

### BACKGROUND OF THE INVENTION

1. Field of the Invention

The invention relates to methods of treating renal cell carcinoma. More specifically, this invention relates to methods of treating renal carcinoma using fully human monoclonal antibodies against the human epidermal growth factor receptor (EGFr).

2. Description of the Related Art

Renal cell carcinoma, or cancer of the kidney, is a serious and often fatal disease that is resistant to traditional forms of treatment. In recent years there have been approximately 12,000 kidney cancer associated deaths annually and approximately 31,000 new cases of kidney cancer in the United States annually. Renal carcinoma is characterized by a lack of early warning signs, therefore, the advanced form of the disease, or metastatic form, is usually found in a patient upon diagnosis. The overall relapse rate following radical nephrectomy is high, but if the localized disease is detected at an early stage, surgery provides the only possibly curative option.

Unfortunately, metastatic renal carcinoma is highly resistant to systemic therapies, thus therapeutic options for patients with advanced forms of the disease are very limited. Most patients fail to respond to current anti-tumor treatment, such as radiation, chemotherapy, and surgery, both when administered singularly and in combination. Despite advancements in surgical techniques and the use of immunotherapy agents most people with metastatic renal carcinoma die within one year of diagnosis. More effective and less toxic therapies for renal carcinoma are urgently needed.

In view of this problem, researchers have begun to explore the treatment potential of immunomodulators. Human trials involving immunotherapy using interleukin-2 and alpha interferon have been conducted but have yet to yield an effective treatment solution in most patients. Scientists have begun studying the role of epidermal growth factor (EGF), which binds to the EGF receptor and provides intracellular signals crucial to tumor formation and survival. These signals have been found to initiate several tumor promoting responses, such as cell invasion and metastasis, and the formation of new blood vessels through angiogenesis, and tumor resistance to conventional therapies. However, prior studies of the receptor biology would not directly lead to an effective treatment.

One company, ImClone Systems Incorporated, has used a chimeric anti-EGFr antibody known as C225 to treat renal cell cancer. However, only a small percentage of human patients responded to the treatment.

Although companies such as Abgenix, Inc. (Fremont, Calif.) have developed mice which produce fully human antibodies called XenoMouse™, no one has yet found therapeutic antibodies which are therapeutically useful against renal cell cancer. Thus, what is needed in the art is a successful and safe treatment for renal cell cancer.

### SUMMARY

One embodiment of the invention is a method of treating renal carcinoma in a patient by first providing a human patient in need of treatment for renal carcinoma. The patient is then administered with a therapeutically effective amount of a fully human monoclonal antibody ABX-EGF, or antigen binding fragments thereof, capable of binding the epidermal growth factor receptor (EGFr). This administration results in an effective treatment for the renal carcinoma. In an alternate preferred embodiment, the method further includes employing dose related skin rash is used as a surrogate biomarker.

Yet another embodiment is a kit for treatment of renal carcinoma in a human patient. The kit includes a fully human monoclonal antibody ABX-EGF that binds to the epidermal growth factor receptor (EGFr) in a pharmaceutically acceptable carrier and instructions for administering to said human patient a therapeutically effective dose of said fully human antibody.

Another embodiment is an article of manufacture comprising a container, a composition contained therein, and a package insert or label. The package insert or label indicates that the composition can be used to treat renal carcinoma characterized by cancer cells expressing epidermal growth factor receptor (EGFr). In addition, the composition comprises the fully human monoclonal antibody ABX-EGF, or antigen binding fragments thereof.

For purposes of summarizing the invention and the advantages achieved over the prior art, certain objects and advantages of the invention have been described herein above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

All of these embodiments are intended to be within the scope of the invention herein disclosed. These and other embodiments of the present invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

FIG. 1A is a representation of effect of ABX-EGF and isotype-matched control antibody PK16.3.1 on EGFr phosphorylation as determined by ELISA after exposure to EGFr for 1 hour.

FIG. 1B is a representation of effect of ABX-EGF and isotype-matched control antibody PK16.3.1 on EGFr phosphorylation as determined by ELISA after exposure to EGFr for 2 hours.

FIG. 2 is a graph of a clonogenic assay showing mean tumor colonies±SEM when human renal carcinoma Caki-1 and Caki-2 cells were seeded and treated with ABX-EGF or control antibody PK 16.3.1.

FIG. 3 is a graph showing the effect of ABX-EGF on the growth of human renal carcinoma SK-RC-29 in xenograft models in mice.

FIG. 4 is a graph showing the effect of ABX-EGF on the growth of human renal carcinoma SK-RC-29 in xenograft models in mice.

FIG. 5 is a graph showing the effect of ABX-EGF on the growth of human renal carcinoma Caki-1 in xenograft models in mice.

FIG. 6 is a graph showing the effect of ABX-EGF on the growth of human renal carcinoma Caki-2 in xenograft models in mice.

FIG. 7 is a graph of the pharmacokinetics of ABX-EGF in patients treated with different doses of ABX-EGF.

FIG. 8 is a graph showing the incidence of patients who developed skin rash relative to dose of ABX-EGF.

FIG. 9 is a bar graph showing the intensity of skin rash by dose in patients treated with ABX-EGF.

FIG. 10 is a bar graph of tumor response by dose in patients treated with ABX-EGF.

### DETAILED DESCRIPTION

One embodiment of the invention is a method of treating renal carcinoma by treating a human patient with fully human monoclonal antibodies against the EGFr. However, this invention is not limited to full-length antibodies. For example, antigen binding fragments or Fab′ fragments of fully human anti-EGFr antibodies are also within the scope of the invention. Methods of using these fragments and full-length EGFr antibodies as renal carcinoma treatments in monotherapy, combined therapies, treatment kits, and in articles of manufacture are also provided.

A. Definitions

Unless otherwise required by context, singular terms shall include pluralities and plural terms shall include the singular.

“Native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Chothia et al. J. Mol. Biol. 186:651 (1985; Novotny and Haber, Proc. Natl. Acad. Sci. U.S.A. 82:4592 (1985); Chothia et al., Nature 342:877-883 (1989)).

The term “antibody” refers to both an intact antibody and an antigen binding fragment thereof which competes with the intact antibody for specific binding. “Antigen binding fragment thereof” refers to a portion or fragment of an intact antibody molecule, wherein the fragment retains the antigen-binding function. Binding fragments are produced by recombinant DNA techniques, or by enzymatic or chemical cleavage of intact antibodies such as papain. Binding fragments include Fab, Fab′, F(ab′)₂, Fv, single-chain antibodies (“scFv”), Fd′ and Fd fragments. Methods for producing the various fragments from monoclonal antibodies are well known to those skilled in the art (see, e.g., Pluckthun, 1992, Immunol. Rev. 130:151-188). An antibody other than a “bispecific” or “bifunctional” antibody is understood to have identical binding sites. An antibody substantially inhibits adhesion of a receptor to a ligand when an excess of antibody reduces the quantity of receptor bound to ligand by at least about 20%, 40%, 60% or 80%, or more (as measured in an in vitro competitive binding assay).

An “isolated” antibody is one which has been identified and separated and/or recovered from a component of its natural environment. Contaminant components of a natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and terminal or internal amino acid sequence by use of a spinning cup sequenator, or (3) to homogeneity by SDS-PAGE under reducing or nonreducing conditions using Coomassie blue or, more preferably, silver stain. An isolated antibody includes an antibody in situ within recombinant cells since at least one component of the antibody's natural environment will not be present. Ordinarily, however, isolated antibodies will be prepared by at least one purification step.

Antibody-dependent cell-mediated cytotoxicity” and “ADCC” refer to a cell-mediated reaction in which non-specific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. Fe expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362, or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1988).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al (1991). The constant domains are not involved directly in binding an antibody to an antigen, but exhibit various effector functions, such as participation of the antibody in antibody-dependent cellular toxicity.

“Fv” is the minimum antibody fragment which contains a complete antigen-recognition and binding site. In a two-chain Fv species, this region consists of a dimer of one heavy- and one light-chain variable domain in tight, non-covalent association. In a single-chain Fv species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-62 (L2), and 89-97 (L3) in the light chain variable domain and 31-55 (H1), 50-65 (H2) and 95-102 (H3) in the heavy chain variable domain; Kabat et al., Sequences of Proteins of Immunological Interest, 5^{th} Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 ((H1), 53-55 (H2) and 96-101 (H3) in the heavy chain variable domain; Chothia and Lesk J. Mol. Biol 196:901-917 (1987)). “Framework Region” or “FR” residues are those variable domain residues other than the hypervariable region residues as herein defined.

The term “complementarity determining regions” or “CDRs” when used herein refers to parts of immunological receptors that make contact with a specific ligand and determine its specificity. The CDRs of immunological receptors are the most variable part of the receptor protein, giving receptors their diversity, and are carried on six loops at the distal end of the receptor's variable domains, three loops coming from each of the two variable domains of the receptor.

The term “epitope” is used to refer to binding sites for (monoclonal or polyclonal) antibodies on protein antigens.

The term “amino acid” or “amino acid residue,” as used herein refers to naturally occurring L amino acids or to D amino acids as described further below with respect to variants. The commonly used one- and three-letter abbreviations for amino acids are used herein (Bruce Alberts et al., Molecular Biology of the Cell, Garland Publishing, Inc., New York (4th ed. 2002)).

The term “disease state” refers to a physiological state of a cell or of a whole mammal in which an interruption, cessation, or disorder of cellular or body functions, systems, or organs has occurred.

The term “treat” or “treatment” refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

A “disorder” is any condition that would benefit from treatment of the present invention. This includes chronic and acute disorders or disease including those pathological conditions which predispose the mammal to the disorder in question. A non-limiting example of a disorder to be treated herein includes renal cell carcinoma (RCC).

“Mammal” for purposes of treatment refers to any animal classified as a mammal, including humans, domestic and farm animals, and zoo, sports, or pet animals, such as dogs, horses, cats, cows, etc. Preferably, the mammal is human.

The term “antineoplastic agent” is used herein to refer to agent(s) that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents. Antineoplastic agents include standard chemotherapuctic and biotherapuetic agents. An “antineoplastic therapy” is the therapeutic administration of one or more antineoplastic agents.

A treatment which exhibits “substantially stable pharmacokinetics” is a treatment which, when administered at a desired dosage, remains in the patient's bloodstream over the course of approximately a month. The treatment preferably provides substantially consistent exposure of the treatment to the target cells.

In accordance with the present invention a method of using fully human monoclonal antibodies is provided for the treatment of renal cell carcinoma. In connection with this treatment, three clinical pathways of combined therapy, monotherapy and low dosage therapy appear to offer distinct potentials for clinical success:

“Combined therapy” refers to the treatment of renal cell carcinoma in which patients would be treated with antibodies in accordance with the present invention in combination with an antineoplastic agent (e.g. a chemotherapuetic or biotherapeutic agent) and/or radiation therapy. Renal cell carcinoma is treated under protocol by the addition of anti-EGFr antibodies to standard first and second line therapy. Protocol designs address the effectiveness as assessed by reduction in tumor mass as well as the ability to reduce usual doses of standard antineoplastic therapy. These dosage reductions will allow additional and/or prolonged therapy by reducing dose-related toxicity of the chemotherapeutic agent. In alternate combined therapy embodiments, an anti-EGFr antibody, or fragment thereof is conjugated to a toxin or other treatment drug, in order to increase the effectiveness of a renal carcinoma treatment.

“Monotherapy” refers to the treatment of renal cell carcinoma by administering anti-EGFr antibodies to patients without an accompanying antineoplastic agent.

Moreover, renal cell carcinoma antibody therapy, as a monotherapy, was successful in clinical trials in stabilizing or reducing tumor growth using anti-EGFr antibodies as described below. The results demonstrate that the antibodies described herein are efficacious as a monotherapy, in addition to combination therapy with an antineoplastic agent against renal cell carcinoma.

Furthermore, ABX-EGF antibodies (Abgenix, Inc., Fremont, Calif.) appear efficacious for treating renal carcinoma at lower doses than observed with prior art antibodies.

B. Methods for Carrying Out the Invention

Embodiments of the invention relate to antibodies directed against renal cell carcinoma and methods and means for making and using such antibodies. One embodiment of the present invention provides antibodies that affect the ability of a renal cell carcinoma to progress.

1. Generation of Anti-EGFr Antibodies

A description follows as to exemplary techniques for the production of the antibodies used in accordance with the present invention.

(i) Monoclonal Antibodies

Monoclonal antibodies may be made using the hybridoma method first described by Kohler et al., Nature 256: 495 (1975), or may be made by recombinant DNA methods (U.S. Pat. No. 4,816,567).

In the hybridoma method, a mouse or other appropriate host animal, such as a hamster or macaque monkey, is immunized as herein above described to elicit lymphocytes that produce or are capable of producing antibodies that will specifically bind to the protein used for immunization. Alternatively, lymphocytes may be immunized in vitro. Lymphocytes or, more preferably, lymphocytes enriched for B cells then are fused with myeloma cells by an electrocell fusion process or by using a suitable fusing agent, such as polyethylene glycol, to form a hybridoma cell (Goding, Monoclonal Antibodies: Principles and Practice, pp.59-103, [Academic Press, 1996]).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOP-21 and MC.-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol. 133: 3001 (1984); Brodeur et al., Monoclonal Antibody Production Techniques and Applications, pp. 51-63, Marcel Dekker, Inc., New York, [1987]).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis of Munson et al., Anal. Biochem. 107: 220 (1980).

After hybridoma cells are identified that produce antibodies of the desired specificity, affinity, and/or activity, the cells may be subcloned by limiting dilution procedures and grown by standard methods (Goding, Monoclonal Antibodies: Principles and Practice, pp.59-103, Academic Press, 1996). Suitable culture media for this purpose include, for example, DMEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by covalently joining to the immunoglobulin coding sequence all or part of the coding sequence for a non-immunoglobulin polypeptide. In that manner, “chimeric” or “hybrid” antibodies are prepared that have the binding specificity of the monoclonal antibodies discussed herein.

Typically such non-immunoglobulin -polypeptides are substituted for the constant domains of an antibody of the invention, or they are substituted for the variable domains of one antigen-combining site of an antibody of the invention to create a chimeric bivalent antibody comprising one antigen-combining site having specificity for the EGFr and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

(ii) Human Antibodies

Attempts to use the same technology for generating human mAbs have been hampered by the lack of a suitable human myeloma cell line. The best results were obtained using heteromyelomas (mouse x human hybrid myelomas) as fusion partners (Kozbor, J. Immunol. 133: 3001 (1984); Brodeur, et al., Monoclonal Antibody Production Techniques and Applications, pp.51-63, Marcel Dekker, Inc., New York, 1987). Alternatively, human antibody-secreting cells can be immortalized by infection with the Epstein-Barr virus (EBV). However, EBV-infected cells are difficult to clone and usually produce only relatively low yields of immunoglobulin (James and Bell, J. Immunol. Methods 100: 5-40 [1987]). In the future, the immortalization of human B cells might possibly be achieved by introducing a defined combination of transforming genes. Such a possibility is highlighted by a recent demonstration that the expression of the telomerase catalytic subunit together with the SV40 large T oncoprotein and an oncogenic allele of H-ras resulted in the tumorigenic conversion of normal human epithelial and fibroblast cells (Hahn et al., Nature 400: 464-468 [1999]).

It is now possible to produce transgenic animals (e.g., mice) that are capable, upon immunization, of producing a repertoire of human antibodies in the absence of endogenous immunoglobulin production (Jakobovits et al., Nature 362: 255-258 [1993]; Lonberg and Huszar, Int. Rev. Immunol. 13: 65-93 [1995]; Fishwild et al., Nat. Biotechnol. 14: 845-851 [1996]; Mendez et al., Nat. Genet. 15: 146-156 [1997]; Green, J. Immunol. Methods 231: 11-23 [1999]; Tomizuka et al., Proc. Natl. Acad. Sci. USA 97: 722-727 [2000]; reviewed in Little et al., Immunol. Today 21: 364-370 [2000]). For example, it has been described that the homozygous deletion of the antibody heavy chain joining region (J_{H}) gene in chimeric and germ-line mutant mice results in complete inhibition of endogenous antibody production (Jakobovits et al., Proc. Natl. Acad. Sci. USA 90: 2551-2555 [1993]). Transfer of the human germ-line immunoglobulin gene array in such germ-line mutant mice results in the production of human antibodies upon antigen challenge (Jakobovits et al., Nature 362: 255-258 [1993]).

Mendez et al. (Nature Genetics 15: 146-156 [1997]) have generated a line of transgenic mice designated as “XenoMouse® II” that, when challenged with an antigen, generates high affinity fully human antibodies. This was achieved by germ-line integration of megabase human heavy chain and light chain loci into mice with deletion into endogenous J_{H} segment as described above. The XenoMouse® II harbors 1,020 kb of human heavy chain locus containing approximately 66 V_{H} genes, complete D_{H} and J_{H} regions and three different constant regions (μ, δ and γ), and also harbors 800 kb of human K locus containing 32 Vκ genes, Jκ segments and Cκ genes. The antibodies produced in these mice closely resemble that seen in humans in all respects, including gene rearrangement, assembly, and repertoire. The human antibodies are preferentially expressed over endogenous antibodies due to deletion in endogenous J_{H} segment that prevents gene rearrangement in the murine locus.

Such XenoMice may be immunized with an antigen of particular interest, such as the EGFr. Sera from such immunized animals may be screened for antibody-reactivity against the initial antigen. Lymphocytes may be isolated from lymph nodes or spleen cells and may further be selected for B cells by selecting for CD138-negative and CD19+ cells. In one aspect, such B cell cultures (BCCs) may be fused to myeloma cells to generate hybridomas as detailed above. In another aspect, such B cell cultures may be screened further for reactivity against the initial antigen, preferably the EGFr protein. Such screening includes ELISA with EGFr-His protein, a competition assay with known antibodies that bind the antigen of interest, such as antibody G250, and in vitro binding to transiently transfected CHO cells expressing full length EGFr. Such screens are further described in the Examples. To isolate single B cells secreting antibodies of interest, an EGFr-specific hemolytic plaque assay is performed. Cells targeted for lysis are preferably sheep red blood cells (SRBCs) coated with the EGFr antigen. In the presence of a B cell culture secreting the immunoglobulin of interest and complement, the formation of a plaque indicates specific EGFr-mediated lysis of the target cells. The single antigen-specific plasma cell in the center of the plaque can be isolated and used for isolation of mRNA.

Using reverse-transcriptase PCR, the DNA encoding the variable region of the antibody secreted can be cloned. Such cloned DNA can then be further inserted into a suitable expression vector, preferably a vector cassette such as a pcDNA, more preferably such a pcDNA vector containing the constant domains of immunglobulin heavy and light chain. The generated vector can then be transfected into host cells, preferably CHO cells, and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ precipitation and electroporation. Successful transfection is generally recognized when any indication of the operation of this vector occurs within the host cell.

In a further embodiment, the phage display technology can be used to produce human antibodies and antibody fragments in vitro, from immunoglobulin variable (V) domain gene repertoires from unimmunized donors (McCafferty et al., Nature 348: 552-553 [1990]; reviewed in Kipriyanov and Little, Mol. Biotechnol. 12: 173-201 [1999]; Hoogenboom and Chames, Immunol. Today 21: 371-378 [2000]). According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats (reviewed in Johnson and Chiswell, Current Opinion in Structural Biology 3: 564-571 [1993)]; Winter et al., Annu. Rev. Immunol. 12: 433-455 [1994]; Dall'Acqua and Carter, Curr. Opin. Struct. Biol. 8: 443-450 [1998]; Hoogenboom and Chames, Immunol. Today 21: 371-378 [2000]). Several sources of V-gene segments can be used for phage display. Clackson et al., (Nature 352: 624-628 [1991]) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222: 581-597 (1991), or Griffiths et al., EMBO J. 12: 725-734 (1993).

In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (Marks et al., Bio/Technol. 10: 779-783 [1992]). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the nM range. A strategy for making very large phage antibody repertoires (also known as “the mother-of-all libraries”) has been described by Waterhouse et al., Nucl. Acids Res. 21: 2265-2266 (1993), and the isolation of a high affinity human antibody directly from such large phage library is reported by Griffiths et al., EMBO J. 13: 3245-3260 (1994). Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e., the epitope governs (imprints) the choice of partner. When the process is repeated in order to replace the remaining rodent V domain, a human antibody is obtained (see PCT patent application WO 93/06213, published Apr. 1, 1993). Unlike traditional humanization of rodent antibodies by CDR grafting, this technique provides completely human antibodies, which have no framework or CDR residues of rodent origin.

C. Dose and Route of Administration

“Effective doses” include doses of 0.1 to 10 mg/kg, more preferably 1.0 to 5.0 mg/kg and most preferably approximately 0.5 mg/kg to 2.5 mg/kg, preferably administered either weekly, every two (2) weeks or every three (3) weeks. In a clinical study described below (Example 1), one patient had a partial response of 50% tumor shrinkage having received 4 doses of 1.5 mg/kg of anti-EGFr antibody ABX-EGF over the course of 42 days. Doses can be administered weekly, bi-weekly, or any other effective time period determined by those of skill in the art. In another clinical study (Example 3), out of 88 patients, 56% (49 patients) exhibited tumor shrinkage or a stable disease state, while 6% (5 patients) exhibited tumor shrinkage receiving dosages ranging from 1.0 mg/kg to 2.5 mg/kg per week. Based on the disclosure contained herein, the skilled artisan would appreciate that higher or lower doses could also be effective. For example, it would be expected that dosages ranging from 0.5 mg/kg to 5.0 mg/kg would also be effective in certain patients. In addition, it would also be expected that the dosage ranges disclosed herein would also be effective when administered once every two (2) to three (3) weeks.

Antibodies in accordance with one embodiment of the present invention have a four (4) to five (5) times higher affinity for EGFr than prior art antibodies, such as C225 (C225 affinity 2×10⁻¹⁰ vs ABX-EGF affinity 5×10⁻¹¹). For example, antibodies for use in accordance with preferred embodiments of the present invention (and particularly the E2.5 and E7.6.3 versions of ABX-EGF) have significantly higher affinities (E2.5: 1.6×10⁻¹¹ M; E7.6.3: 5.7×10⁻¹¹ M). Antibodies for use in accordance with preferred embodiments also preferably block ligand binding and, in addition, preferably inhibit both EGF-dependent EGFr phosphorylation and tumor cell proliferation. One preferred embodiment employs a fully human IgG2k antibody which binds to EGFr with an affinity of about KD=50 pM. Certain preferred embodiments are efficacious in monotherapy while other preferred embodiments are efficacious in combination therapy, e.g. or conjugated to a toxin or administered with an antineoplastic agent against renal cell carcinoma as described below.

Furthermore, the ABX-EGF antibody appears efficacious at lower doses than with prior art antibodies which were typically administered in doses ranging from 5 to 400 mg/m². Further, antibodies in accordance with the present invention are fully human antibodies and, thus, have relatively slow clearance from the blood. Accordingly, it is expected that dosing in patients with antibodies in accordance with the invention can be lower, perhaps in the range of dosing rates of 50 to 300 mg/m², and still remain efficacious. Dosing in mg/m², as opposed to the conventional measurement of dose in mg/kg, is a measurement based on surface area and is a convenient dosing measurement that is designed to include patients of all sizes from infants to adults.

“Therapeutically effective delivery route” refers to any treatment delivery route which effectively delivers the fully human monoclonal antibodies to the target tumor so that the antibodies can bind EGFr without causing unacceptable side effects. Two distinct delivery approaches are expected to be useful for the delivery of antibodies in accordance with the invention. Conventional intravenous delivery will presumably be the standard delivery technique for the majority of tumors. However, in connection with tumors in the peritoneal cavity, intraperitoneal administration may prove favorable for obtaining high doses of antibody at the tumor and to minimize antibody clearance. In a similar manner certain solid tumors possess vasculature that is appropriate for regional perfusion. Regional perfusion will allow the obtention of a high dose of the antibody at the site of a tumor and will minimize short term clearance of the antibody. In addition, both subcutaneous delivery and intramuscular delivery could also be effectively employed.

### EXAMPLES

The following examples, including the experiments conducted and results achieved are provided for illustrative purpose only and are not to be construed as limiting upon the present invention.

### Example 1

A renal cell cancer patient was given intravenous administration of 1.5 mg/kg of anti-EGFr antibody ABX-EGF (Abgenix, Inc., Fremont, Calif.). The patient reported improvements in symptoms after only 4 weekly doses as shown by a CT scan of the patient's chest after four weeks of treatment. After 42 days elapsed since the treatment began, the patient exhibited a greater than 50% shrinkage of the tumor. The tumor shrinkage was documented using CT imaging. This demonstrated that the ABX-EGF antibody was effective for reducing the size of a metastatic renal carcinoma, and thus can provide a treatment for renal cell carcinoma.

### Example 2

Studies of ABX-EGF monotherapy in metastatic human renal carcinomas in athymic mice will be discussed below. These studies were conducted to determine whether EGFr was overexpressed on the surface of certain types of human renal cell carcinoma cells and, also, to determine whether antibodies against EGFr, such as ABX-EGF, inhibited EGFr autophosphorylation.

A. Materials and Methods:

1. Cell Culture

Three human renal carcinoma cell lines SK-RC-29 (renal carcinoma), Caki-1 (metastatic renal clear cell carcinoma), and Caki-2 (primary renal clear cell carcinoma) were chosen for the study. Human renal cancer cell lines Caki-1, Caki-2 were purchased from the American Type Culture Collection (ATCC, Rockville, Md.). SK-RC-29 was provided by the Ludwig Institute for Cancer Research. Caki-1 and Caki-2 cells were routinely maintained in McCoy's 5A medium supplemented with 10% fetal bovine serum (FBS), SK-RC-29 cells were grown in Dulbecco's Eagle medium (DMEM) with 10% FBS.

2. Determination and Quantitation of EGFr on Renal Tumor Cell Line

Caki-1, Caki-2 and SK-RC-29 cells (0.1×10⁶) were stained with ABX-EGF or human IgG2 isotype-matched control followed by secondary staining with FITC-conjugated goat-anti-human IgG antibody (Caltage Calif.). The EGFr number was quantitated by Quantum Simply Cellular Microbeads (Flow Cytometry Standards Corporation).

3. Inhibition of EGFr Phosphorylation Assay

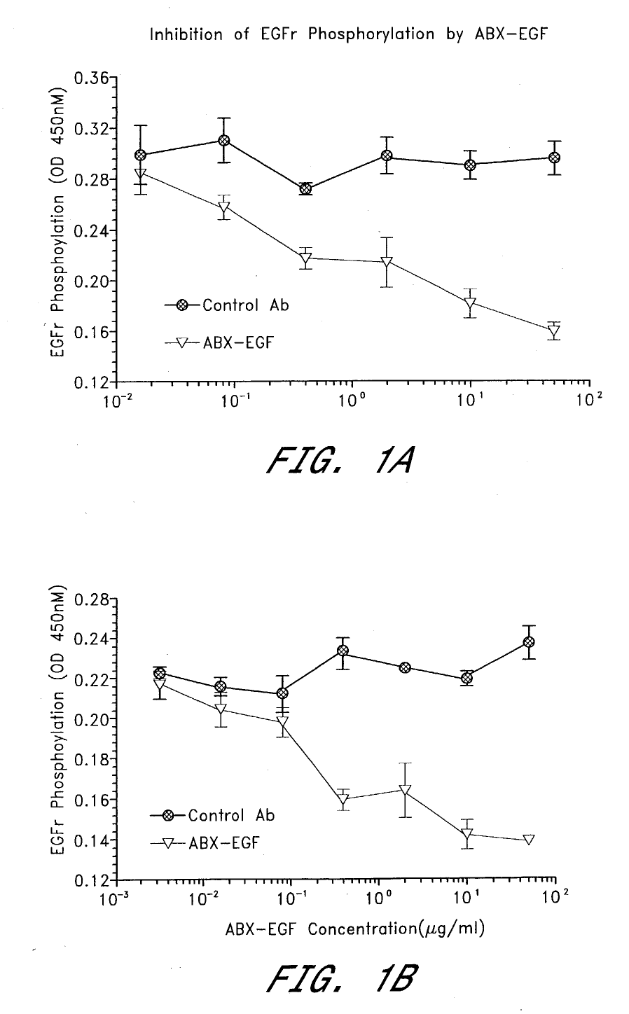

Caki-1, Caki2 and SK-RC-29 cells were seeded 1.5×10⁵/well into 96-well plates overnight. The plates were washed and replaced with serum free medium containing EGF (Sigma) 200 ng/ml and with ABX-EGF at 25 μg/ml for 1, 2, 4 and 24 hours. The cells were then lysed. The EGFr phosphorylation was measured by an ELISA using anti-EGFr Ab and anti-EGFr phosphotyrosine antibody (isotype-matched control). The results after exposure to EGFr for 1 or 2 hours are shown FIGS. 1A and 1B respectively. The baseline EGFr phosphorylation of cells only was 0.16. As shown, treatment with ABX-EGF significantly reduced EGFr phosphorylation in a concentration dependent manner.

4. Clonogenic Assay

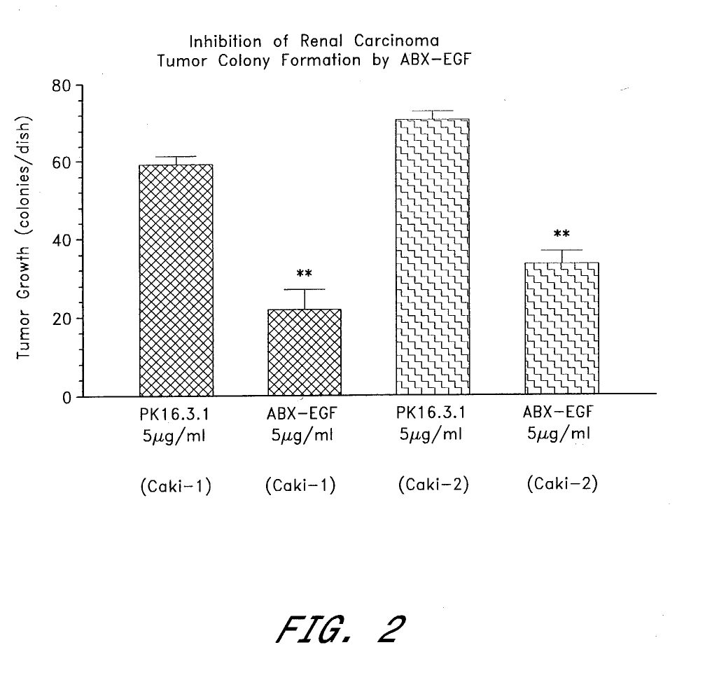

Human renal carcinoma Caki-1 and Caki-2 cells were seeded at 1×10⁴/dish and cultured in the presence of or control antibody PK 16.3.1 or 5 μg/ml of ABX-EGF for 7 days. The dishes were incubated for additional 2 weeks. The tumor cell colonies were stained and counted. Washed and trypsinized single-cell suspensions were plated into 60 mm² culture dish with a total 0.2×10³ cells per dish. After 14 days incubation with a medium change weekly, the colonies were stained with 5 mM methylene blue and counted. The results shown in FIG. 2 represents mean tumor size±SEM.

5. Mouse Xenografts

BALB/c male nude mice (6-8 weeks of age) were implanted subcutaneously with 5×10⁶ Caki-1, Caki-2 or SK-RC-29 cells/mouse. Tumors were measured with vernier calipers. Tumor volume was calculated by the formula: length×width×height×π/6. Mice with established tumors were randomly divided into treatment groups (n=10). ABX-EGF was injected intraperitoneally twice a week for three weeks.

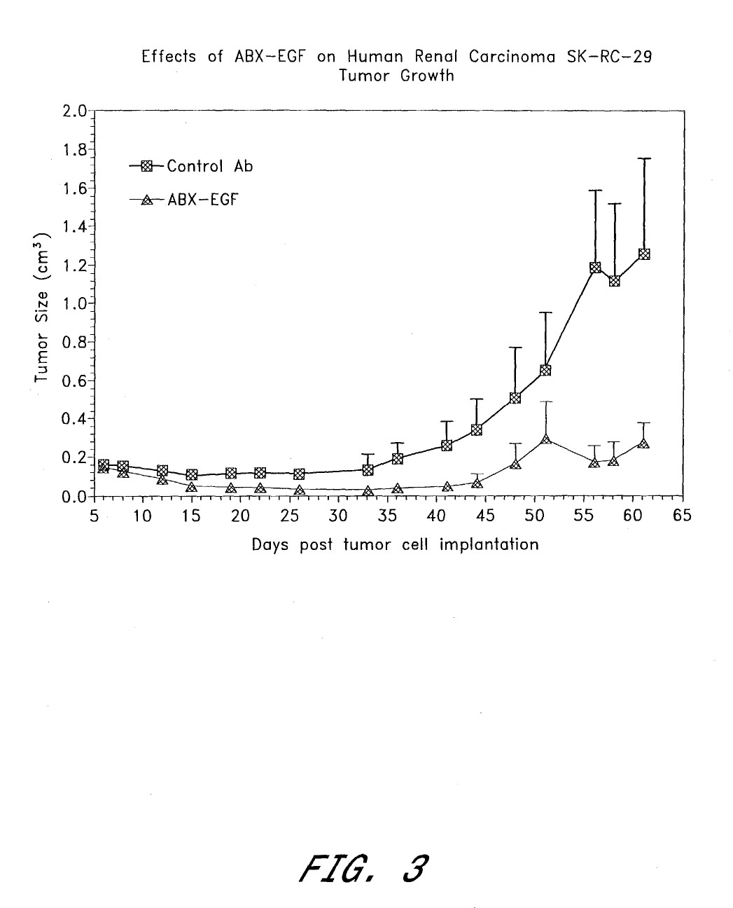

In order to determine the effect ABX-EGF on human renal carcinoma SK-RC-29 cells, the SK-RC-29 cells (5×10⁶) were injected subcutaneously into nude mice (n=10). ABX-EGF (1.0 mg) or PBS control was administrated on day 6 intraperitoneally twice a week for 3 weeks. The resulting data shown in FIG. 3 represent the mean of tumor size±SEM. As shown, ABX-EGF was effective in reducing the size of the tumor over time.

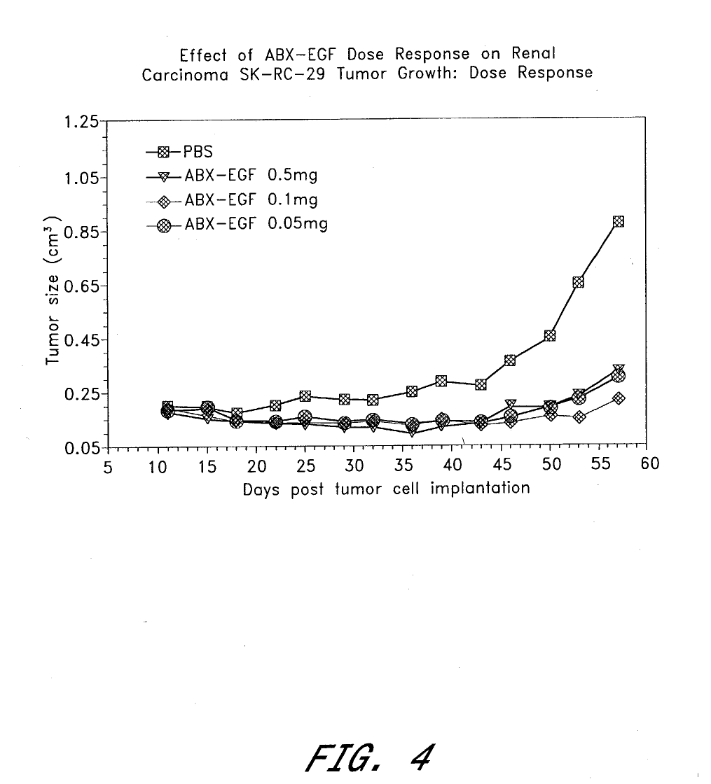

In order to determine the dose response of ABX-EGF on human renal carcinoma SK-RC-29 cells, the SK-RC-29 cells (5×10⁶) were injected subcutaneously into nude mice (n=10) at day 0. When the tumor size reached to approximately 0.2 cm³, ABX-EGF or PBS was administrated intraperitoneally for 3 weeks. The resulting data shown in FIG. 4 represent the mean of tumor size±SEM. As shown, ABX-EGF was effective in reducing the size of the tumor over time.

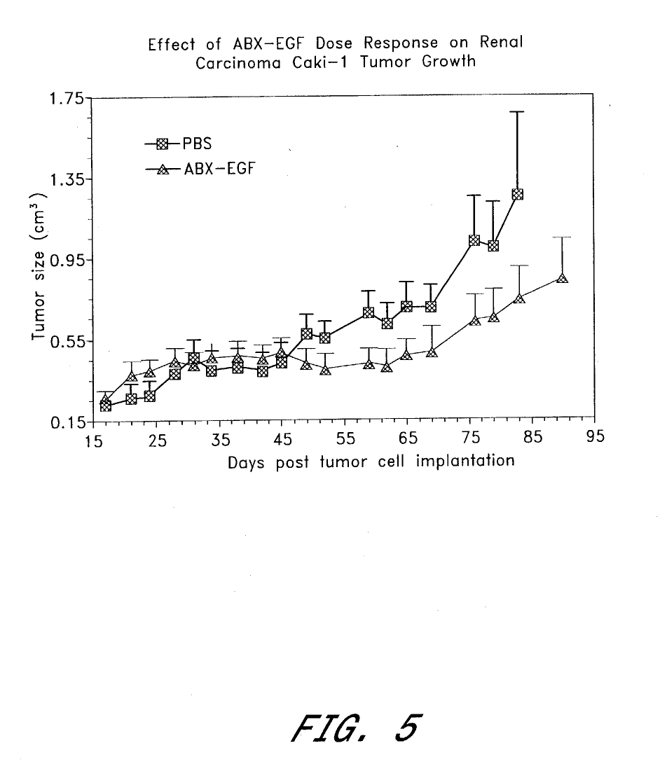

In order to determine the effect of ABX-EGF on human renal carcinoma Caki-1 cells, the Caki-1 cells (5×10⁶) were injected subcutaneously into nude mice at day 0. When the tumor sizes reached to approximately 0.25 cm³, at day 16, ABX-EGF (1 mg) or PBS was administrated intraperitoneally twice a week for 3 weeks. The resulting data shown in FIG. 5 represent the mean of tumor size±SEM.

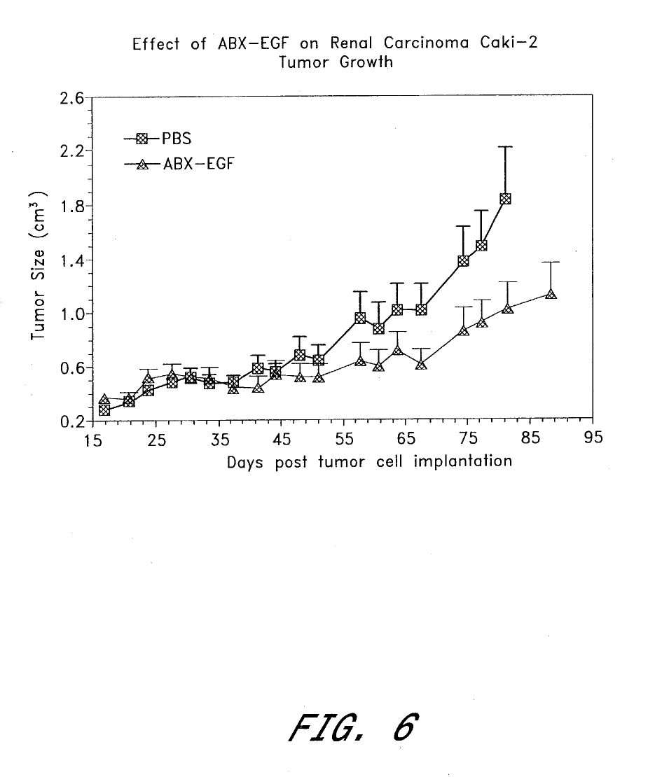

In order to determine the dose response of antibodies against EGFr on human renal carcinoma Caki-2 cells, Caki-2 cells (5×10⁶) were inoculated subcutaneously into nude mice (n=10) at day 0. Tumor sizes were measured twice a week. When the tumor sizes reached to approximately 0.3 cm³, ABX-EGF (1 mg) or PBS control was administered intraperitoneally twice a week for 3 weeks. The resulting data shown in FIG. 6 data an of tumor size±SEM.

B. Results

EGFr Expression on the Surface of Human Renal Cell Carcinoma Cells

Flow cytometry based analysis demonstrated that all three renal tumor cell lines subjected to this study express significant levels of EGFr as shown in Table 1 below.

TABLE 1

| Cell line | Tumor Type | EGFr # per cell |
|---|---|---|
| Caki-1 | Metastatic RCC | 69,000 |
| Caki-2 | Primary RCC | 258,000 |
| SK-RC-29 | Metastatic RCC | 77,000 |

2. ABX-EGF Inhibited EGFr Autophosphorylation

ABX-EGF inhibited EGFr autophosphorylation in vitro and tumor growth in vivo using SK-RC-29 cells. Accordingly, monotherapy with ABX-EGF resulted in a profound inhibition of tumor growth in the xenograft model. This data suggests that ABX-EGF is an effective monotherapeutic agent for the treatment of human renal cell carcinoma.

### Example 3

A. Introduction

Human patients with renal cancer received multi-dose administration of ABX-EGF in order to assess both the saftety and the clinical effect and, also, to determine the pharmacokinetics. The details of the experiments are presented below.

B. Experimental Design

The trial was multi-dose and open label. Four cohorts, with 21-23 patients per cohort, were administered ABX-EGF, in a sequential dose rising order. Patients in the first cohort each received 1.0 mg/kg per week. In addition, patients in the second cohort received 1.5 mg/kg per week, while those in the third cohort received 2.0 mg/kg per week and those in the fourth cohort received 2.5 mg/kg per week. All four cohorts were administered the above doses for a total of 39 weeks with response assessments every 8 weeks.

C. Patient Population

1. Population Inclusion

Patients participating in this trial all had metastatic renal cell carcinoma. In addition, the patients had received and failed IL-2 or interferon therapy or were unwilling/unable to receive IL-2 or interferon. The patients were selected to have a bi-dimensionally measurable disease and tumor tissue available for diagnostics. Furthermore, the patients had adequate hematologic, renal and hepatic function with an ECOG score of 0 or 1. Table 2 illustrates additional patient disposition data, while Table 3 combines patient demographics data.

TABLE 2
Patient Disposition

| | 1.0 mg/kg | 1.5 mg/kg | 2.0 mg/kg | 2.5 mg/kg | Total N |
|---|---|---|---|---|---|
| | N (%) | N (%) | N (%) | N (%) | (%) |
| Enrolled | 22 | 24 | 25 | 24 | 95 |
| MITT* (≧1 | 22 | 22 | 23 | 21 | 88 |
| dose) | | | | | |
| Completed | 16 (73) | 14 (64) | 9 (39) | 16 (76) | 55 (63) |
| Course 1 | | | | | |
| Remain in | 0 (0) | 0 (0) | 0 (0) | 4 (19) | 4 (5) |
| active | | | | | |
| treatment | | | | | |

*Percentages are based on the number of MITT patients.

TABLE 3
Patient Demographics

| | 1.0 mg/kg | 1.5 mg/kg | 2.0 mg/kg | 2.5 mg/kg | Total |
|---|---|---|---|---|---|
| | N = 22(%) | N = 22(%) | N = 23(%) | N = 21(%) | N = 88 |
| Characteristic | | | | | |
| Mean age | 56 | 57 | 58 | 60 | 58 |
| (years) | | | | | |
| Gender | | | | | |
| Female | 9 (41) | 8 (36) | 4 (17) | 3 (14) | 24 (27) |
| Male | 13 (59) | 14 (64) | 19 (83) | 18 (86) | 64 (73) |
| ECOG | | | | | |
| 0 | 16 (73) | 9 (41) | 15 (65) | 11 (52) | 51 (58) |
| 1 or 2 | 6 (27) | 13 (59) | 8 (35) | 10 (48) | 37 (42) |
| Prior | | | | | |
| Antineoplastic | | | | | |
| Therapy | | | | | |
| 0 | 2 (9) | 1 (5) | 3 (13) | 2 (10) | 8 (9) |
| 1-2 | 13 (59) | 11 (50) | 14 (61) | 10 (48) | 48 (55) |
| ≧3 | 7 (32) | 10 (45) | 6 (26) | 9 (43) | 32 (36) |

2. Population Exclusion

Potential patients who had brain metastasis if not controlled or hypercalcemia were excluded. Those patients who had cancer therapy within 30 days or were treated with prior anti-EGFr agents were not selected. In addition, potential patients with a left ventricular ejection fraction <45% by MUGA Scan or myocardial infarction within were also excluded from the trial population.

TABLE 4
EGFr Over-Expression
(≧10% cells 2+ or 3+ IHC)

| | 1.0 mg/kg | 1.5 mg/kg | 2.0 mg/kg | 2.5 mg/kg | Total |
|---|---|---|---|---|---|
| | N = 22(%) | N = 22(%) | N = 23(%) | N = 21(%) | N = 88 |
| At Least 10% | | | | | |
| at 2+ or 3+ | | | | | |
| N* | 20 | 16 | 20 | 20 | 76 |
| Yes | 19 (95) | 15 (94) | 18 (90) | 17 (85) | 69 (91) |
| No | 1 (5) | 1 (6) | 2 (10) | 3 (15) | 7 (9) |

*Percentages are based on the number of patients with samples evaluated.

D. Results

1. Pharmacokinetics

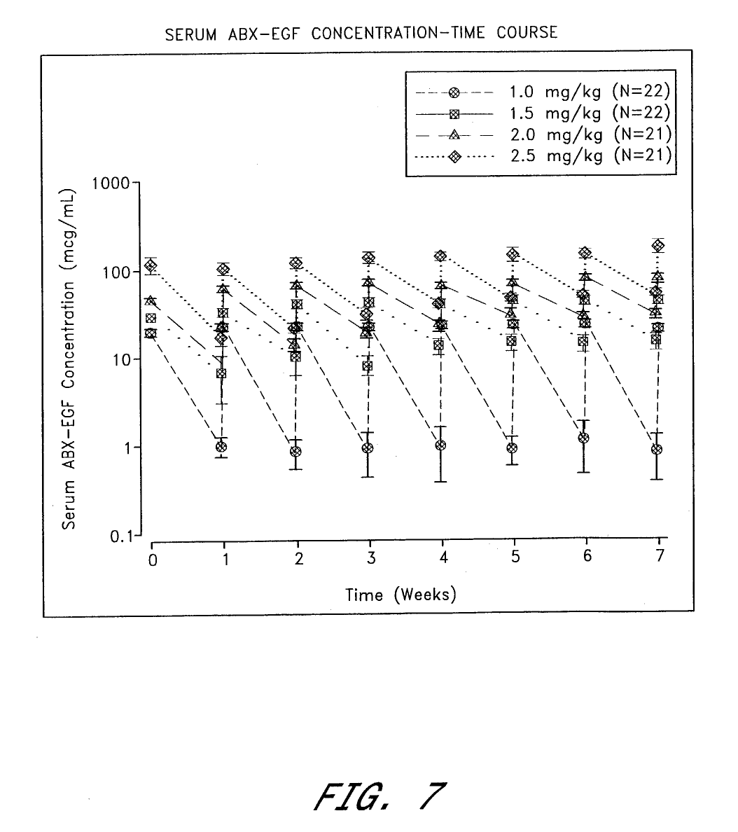

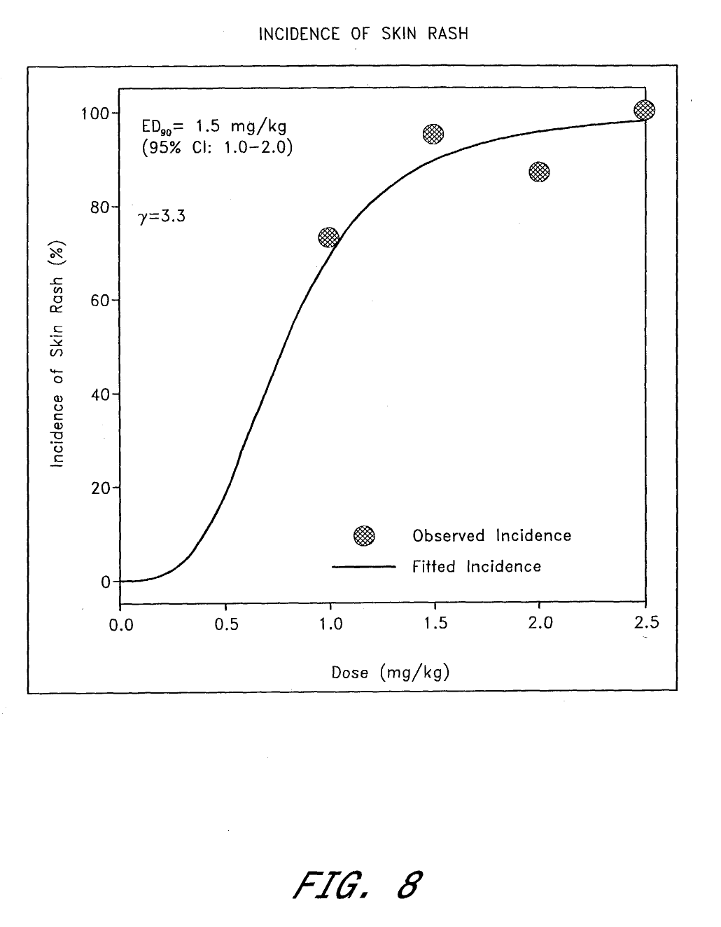

The ABX-EGF treatment was found to offer low intrapatient variability. This low intrapatient variability was supportive of no human anti-human antibody formation (HAHA) (n=69). In addition, no human anti-human antibodies were detected. The pharmacokinetics of the administered treatment are shown in FIG. 7 showing the serum ABX-EGF concentration-time course. Advantageously, the pharmacokinetics of ABX-EGF were found to be substantially stable and revealed consistent exposure of renal carcinomas to ABX-EGF.

2. Incidence of Treatment Emergent Adverse Events

ABX-EGF was found to be generally well tolerated at all dose levels studied and most adverse events have been mild to moderate. These mild to moderate side effects (excluding skin rash) are shown in Table 5. No significant infusion related or allergic reactions were observed. All serious side effects were ultimately resolved, as shown in Table 6.

TABLE 5
Incidence of Treatment Emergent Adverse Events
Grade 2 or Greater and >5% Total Incidence
(Excluding Skin Rash)

| | 1.0 mg/kg | 1.5 mg/kg | 2.0 mg/kg | 2.5 mg/kg | Total |
|---|---|---|---|---|---|
| Event | N = 22(%) | N = 22(%) | N = 23(%) | N = 21(%) | N = 88 |
| Asthenia | 2 (9) | 6 (27) | 4 (17) | 1 (5) | 13 (15) |
| Pain | 2 (9) | 5 (23) | 4 (17) | 0 | 11 (13) |
| Abdominal | 1 (5) | 2 (9) | 2 (9) | 0 | 5 (6) |
| pain | | | | | |
| Back pain | 2 (9) | 6 (27) | 1 (4) | 2 (10) | 11 (13) |
| Constipation | 1 (5) | 2 (9) | 2 (9) | 0 | 5 (6) |
| Cough | 0 | 2 (9) | 1 (4) | 5 (24) | 8 (9) |
| Dyspnea | 1 (5) | 2 (9) | 2 (9) | 4 (19) | 9 (10) |
| Diarrhea | 2 (9) | 1 (5) | 0 (0) | 2 (10) | 5 (6) |

TABLE 6
Incidence of Serious Adverse Events (SAE) Believed to
Be Related to ABX-EGF

| Dose Group | Patient | Event | Outcome | Intensity |
|---|---|---|---|---|
| 1.0 mg/kg | 1 | Dyspnea | Resolved | Moderate |
| | 2 | Diarrhea | Resolved | Severe |
| | 3 | DVT | Resolved | Moderate |
| 1.5 mg/kg | 4 | Vomiting | Resolved | Severe |
| | 5 | Rigors | Resolved | Severe |

*There were no drug related SAEs reported at the 2.0 mg/kg and 2.5 mg/kg dose levels.

3. Incidence of Skin Rash

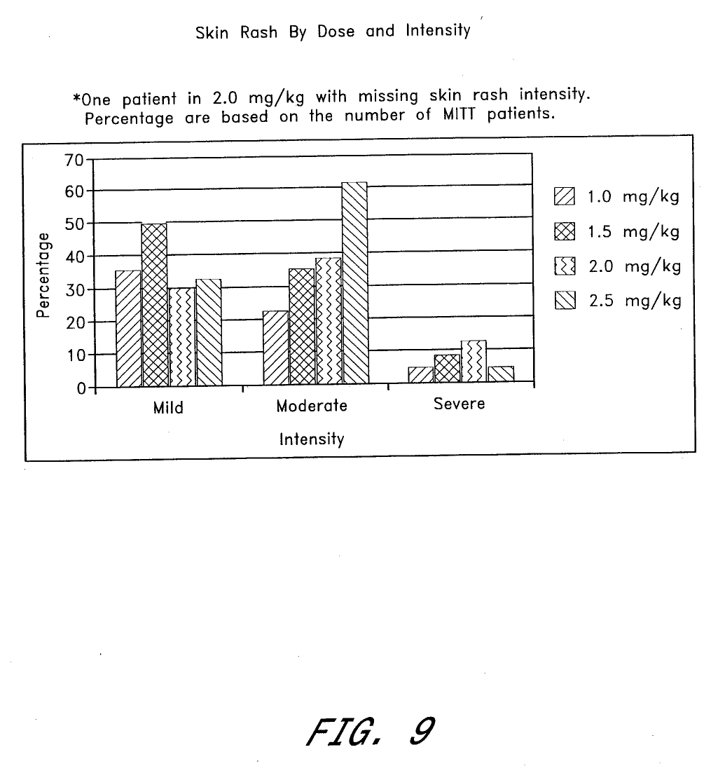

Dose-related acneiform skin rash was found to be a common side effect of the ABX-EGF treatment, with the incidence of skin rash generally increasing with dose as shown in FIG. 9. The pharmacodynamics were found to be that 100% of patients exhibited a skin rash with an increasing dose to 2.5 mg/kg. In addition, the modeled ED₉₀ was found to equal 1.5 mg/kg. The intensity of the skin rash by dose is shown in FIG. 9. Accordingly, the incidence of skin rash in a patient being treated with ABX-EGFr is useful as a surrogate biomarker to determine an effective dose. For example, a therapeutically effective amount of antibodies against EGFr which is effective to treat renal carcinoma in the patient would be partially determined by examining the patient for acnei-form skin rash subsequent to administering a dose or doses. If a skin rash is observed, then a health care practitioner could set the dosage level at the dosage administered prior to the skin rash. If no skin rash were observed, then the health care practitioner could increase the dose until a skin rash were observed. Table 7, showing the rash grading scale used in this example, can be interrelated with the rash severity values shown in Table 6 and FIG. 9 as follows: 1=mild, 2 moderate, and 3=severe.

TABLE 7
Rash Grading CTC v.2
Rash/Desquamation

| | |
|---|---|
| 0 | none |
| 1 | macular or papular eruption or erythema without associates |
| (mild) | symptoms |
| 2 | macular or papular eruption or erythema with pruritus or |
| (moderate) | other associate symptoms covering <50% of body surface or |
| | localized desquamation or other lesions covering <50% of |
| | body surface area |
| 3 | symptomatic generalized erythroderma or macular, papular, or |
| (severe) | vesicular eruption or desquamation covering ≧50% of body |
| | surface area |
| 4 | generalized exfoliative dermatitis or ulcerative dermatitis |

4. Anti-Tumor Activity

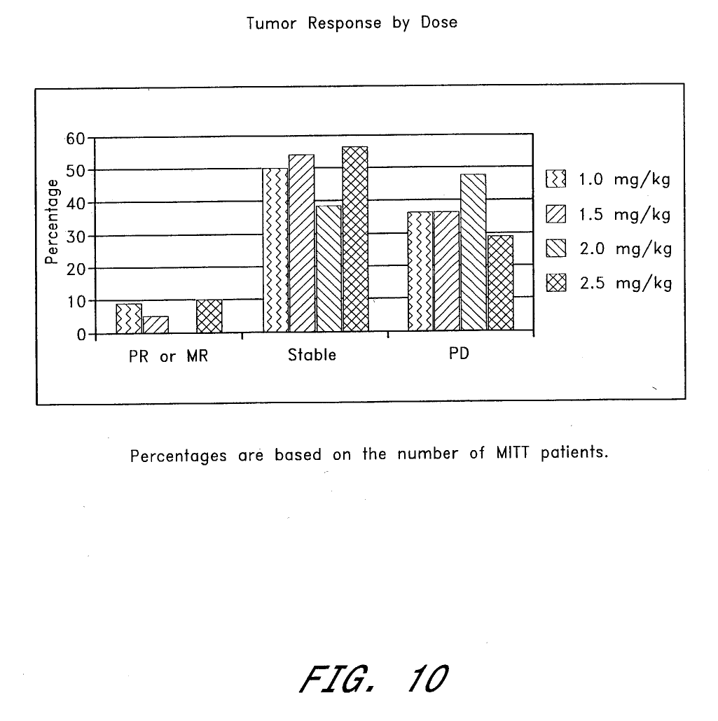

An analysis of the treatment results revealed single agent anti-tumor activity in renal cell cancer. The details of the ABX-EGFr treatment results are shown below in Table 8 and 9 and also in FIG. 10 The time to disease progression is shown in Table 10 with the shown percentages being based on the number of Modified Intent to Treat (MITT) patient. For the purposes of this protocol this analysis population was defined as all patients enrolled in the study who have received at least 1 dose of ABX-EGF.

Tumor response was evaluated using known Response Evaluation Criteria in Solid Tumors (RECIST) techniques as outlined in The Journal of The National Cancer Institute. 92(3):179-81 (Feb. 2, 2000). These RECIST techniques are fully incorporated by reference. A partial response (PR) equals at least a 30% decrease in the sum of the longest diameter (LD) of target lesions, taking as reference the baseline sum LD. A minor response (MR) is approximately between a 20% and a 30% decrease in the sum of the longest diameter (LD) of target lesions, taking as reference the baseline sum LD. As shown in Table 7 and FIG. 10, out of a total of 88 patients, 56% (49 patients) exhibited tumor shrinkage or a stable disease state, while 6% (5 patients) exhibited tumor shrinkage receiving dosages ranging from 1.0 mg/kg to 2.5 mg/kg per week. Based on this data, it would appear that one dose of ABX-EGF in the ranges disclosed herein every 2 to 3 weeks would also be an effective treatment. Accordingly, in those patients listed as having a stable disease state or tumor shrinkage, the ABX-EGF treatment outlined herein was effective for treating their renal cell carcinoma.

TABLE 8
Tumor Response by Dose Level

| Dose (mg/kg) | 1.0 | 1.5 | 2.0 | 2.5 | Total |
|---|---|---|---|---|---|
| N (%) | 22 | 22 | 23 | 21 | 88 |
| PR or MR | 2 (9) | 1 (5) | 0 (0) | 2 (10) | 5 (6) |
| Stable | 11 (50) | 12 (55) | 9 (39) | 12 (57) | 44 (50) |
| PD | 8 (36) | 8 (36) | 11 (48) | 6 (29) | 33 (38) |
| N/A | 1 (5) | 1 (5) | 3 (13) | 1 (5) | 6 (7) |

In relation to the tumor response data shown in Table 8, the Kaplan-Meier median time to disease progression is shown in Table 10.

TABLE 9
Patients with Response

| | | | Time to | No. of | | |
|---|---|---|---|---|---|---|
| | | Re- | Disease | Prior | Nephrec- | Skin |
| Patient | Dose | sponse | Progression | Therapy | tomy | Rash |
| 1 | 1.0 | PR* | 128 | 4 | No | Moderate |
| 2 | 1.0 | MR | 357+ | 1 | Yes | Severe |
| 3 | 1.5 | PR | 295+ | 2 | Yes | Mild |
| 4 | 2.5 | MR | 78 | 4 | Yes | Moderate |
| 5 | 2.5 | PR | 222+ | 4 | Yes | Moderate |

PR = Partial Response
MR = Minor Response
*Primary Tumor Unchanged

TABLE 10
Time to Disease Progression

| | 1.0 mg/kg | 1.5 mg/kg | 2.0 mg/kg | 2.5 mg/kg | Total |
|---|---|---|---|---|---|
| | N = 22(%) | N = 22(%) | N = 23(%) | N = 21(%) | N = 88 |
| Patients with | 18 (82) | 16 (73) | 19 (83) | 15 (71) | 68 (77) |
| DP | | | | | |
| Patients | 4 (18) | 6 (27) | 4 (17) | 6 (29) | 20 (23) |
| Censored* | | | | | |
| Kaplan- | | | | | |
| Meier | | | | | |
| Estimates | | | | | |
| 25^{th} | 53 | 52 | 43 | 57 | 51 |
| Percentile | | | | | |
| Median | 108 | 165 | 53 | 103 | 100 |
| (95% Cl of | (56, 104) | (54, 246) | (46, 100) | (60, 162) | (58, 140) |
| Median**) | | | | | |
| 75^{th} | 161 | 246 | 162 | 162 | 168 |
| Percentile | | | | | |

*Patients who had no disease progression while on study were censored at their last contact date.
**The 95% confidence interval was calculated using the sign test (Brookmeyer and Crowley 1982).

As shown in Table 11, and in Table 9 showing the correlation between pre-therapies and tumor shrinkage response, pre-treating a patient with a preferably systemic therapy, such as one or more antineoplastic therapies, such as biotherapies and/or chemotherapies, prior to administering antibodies against EGFr (or antigen binding fragments thereof), may increase the efficacy of the renal cell carcinoma treatment. Accordingly, a preferred embodiment of the present invention includes pre-treating a patient with one or more biotherapies and/or chemotherapies, preferably 1 to 4 pre-treatments, prior to administering antibodies against EGFr (or antigen binding fragments thereof). Non-limiting examples of pre-treatments include administering interleukin-2, interferon, 5-fluorouracil, thalidomide, dentritic cell vaccine, and/or anti-VEGF monoclonal antibody therapy.

TABLE 11
Heavily Pre-treated RCC Patient Population

| ABX-EGF Dose mg/kg | 1.0 | 1.5 | 2.0 | 2.5 | All |
|---|---|---|---|---|---|
| Number | 22 | 22 | 23 | 21 | 88 |
| Prior Systemic Therapy | | | | | |
| 0 | 5 (23) | 2 (9) | 2 (9) | 1 (5) | 10 (11) |
| 1-2 | 10 (45) | 12 (56) | 11 (61) | 13 (62) | 49 (66) |
| >3 | 7 (32) | 8 (36) | 7 (30) | 7 (33) | 29 (33) |

Accordingly, the above examples show that fully human anti-EGFr antibodies are effective to treat renal carcinoma as a monotherapy. Example 1 demonstrated that the ABX-EGF antibody was effective for reducing the size of a renal carcinoma, and thus can provide a treatment for renal cell carcinoma. Example 2 illustrated that EGFr was overexpressed on the surface of certain types of human renal cell carcinoma cells in athymic mice and, also, that the antibody against EGFr known as ABX-EGF inhibited EGFr autophosphorylation. Example 3 showed the safety, pharmokinetics, and efficacy of ABX-EGF as a renal cell carcinoma treatment and, also, determined preferred dosage ranges in human clinical trials. In addition, Example 3 illustrated that pre-treating a patient with one or more antineoplastic therapies can increase the efficacy of subsequently administering antibodies against EGFr. Furthermore, the results of the above examples show that the same qualities which specifically make the ABX-EGF antibodies against EGFr, highly efficacious as a monotherapy, are equally as advantageous in combined therapies.

The present invention includes effective methods of treating renal carcinoma using the ABX-EGF fully human antibodies against EGFr. The present invention also includes a treatment kit which contains ABX-EGF for the treatment of renal carcinoma and instructions for the effective use of these antibodies.

Incorporation by Reference

All references cited herein, including patents, patent applications, papers, text books, and the like, and the references cited therein, to the extent that they are not already, are hereby incorporated herein by reference in their entirety.

Equivalents

Although this invention has been disclosed in the context of certain preferred embodiments and examples, it will be understood by those skilled in the art that the present invention extends beyond the specifically disclosed embodiments to other alternative embodiments and/or uses of the invention and obvious modifications thereof. Thus, it is intended that the scope of the present invention herein disclosed should not be limited by the particular disclosed embodiments described above, but should be determined only by a fair reading of the claims that follow, including any equivalents thereof.

What is claimed is:

1. A method of treating renal cell carcinoma in a patient comprising:
identifying a human patient in need of treatment for renal cell carcinoma; and
administering to the human patient a therapeutically effective amount of a fully human monoclonal antibody ABX-EGF, or an antigen binding fragment thereof, capable of binding the epidermal growth factor receptor (EGFr), wherein the administering results in an effective treatment for renal cell carcinoma.

2. The method of claim 1, further comprising administering an antineoplastic agent to said patient.

3. The method according to claim 1, wherein the antigen binding fragment is selected from a group consisting of: F (ab′)₂, Fab′, Fab, Fv, scFv, Fd′, and Fd.

4. The method according to claim 1, wherein the fully human monoclonal antibody ABX-EGF, or antigen binding fragment thereof, is conjugated with a treatment agent prior to administrating.

5. The method according to claim 1, wherein the fully human monoclonal antibody ABX-EGF, or an antigen binding fragment thereof, is administered via a therapeutically effective delivery route selected from a group consisting of: intravenous administration, intraperitoneal administration, subcutaneous administration, intramuscular administration and regional perfusion.

6. The method according to claim 1, wherein the fully human monoclonal antibody ABX-EGF, or antigen binding fragment thereof, is recombinant.

7. The method according to claim 1, wherein said therapeutically effective amount is estimated by employing a patient's skin rash as a surrogate biomarker.

8. The method according to claim 1, further comprising determining whether the therapeutically effective amount of the fully human monoclonal antibody ABX-EGF, or antigen binding fragment thereof, is effective to treat renal carcinoma in the patient by examining the patient for acne-form skin rash subsequent to administering the therapeutically effective amount of the fully human monoclonal antibody, or antigen binding fragment thereof.

9. The method according to claim 8, wherein determining whether the therapeutically effective amount of the fully human monoclonal antibody ABX-EGF, or antigen binding fragment thereof, is effective to treat renal carcinoma in the patient further comprises adjusting the administered amount of the fully human monoclonal antibody, or antigen binding fragment thereof, if the patient does not exhibit the skin rash.

10. The method according to claim 8, wherein adjusting the administered amount of the fully human monoclonal antibody ABX-EGF, or antigen binding fragment thereof, comprises increasing the administered amount until the patient does exhibit a skin rash subsequent to administering the therapeutically effective amount of the fully human monoclonal antibody, or antigen binding fragment thereof.

11. The method according to claim 8, further comprises, if the skin rash is observed, continuing to administer the fully human monoclonal antibody ABX-EGF, or antigen binding fragment thereof, at the same amount per week which was administered prior to the onset of the skin rash.

12. The method according to claim 1, further comprising pre-treating the patient with one or more antineoplastic therapies, prior to administering the therapeutically effective amount of the fully human monoclonal antibody ABX-EGF, or antigen binding fragment thereof.

13. The method according to claim 1, wherein the fully human monoclonal antibody ABX-EGF, or antigen binding fragment thereof, administered to the patient which triggers substantially no human anti-human antibody (HAHA) formation.

14. The method according to claim 1, wherein the therapeutically effective amount of the fully human antibody ABX-EGF, or antigen binding fragment thereof, administered to the patient is 0.5 mg/kg to 5 mg/kg.

15. The method according to claim 14, wherein the therapeutically effective amount of the fully human antibody ABX-EGF, or antigen binding fragment thereof, administered to the patient is 0.5 mg/kg to 2.5 mg/kg.

16. The method according to claim 15, wherein the therapeutically effective amount of the fully human antibody ABX-EGF, or antigen binding fragment thereof, administered to the patient is 1 mg/kg to 2.5 mg/kg.

17. The method according to claim 16, wherein the dosage schedule of the therapeutically effective amount of the fully human antibody ABX-EGF, or antigen binding fragment thereof, is one dose per week.

18. The method according to claim 16, wherein the dosage schedule of the therapeutically effective amount of the fully human antibody ABX-EGF, or antigen binding fragment thereof, is one dose given every 2 to 3 weeks.

19. The method according to claim 1, further comprising pre-treating the patient with one or more antineoplastic therapies prior to administering the ABX-EGF antibodies, or antigen binding fragment thereof, in order to increase the efficacy of the ABX-EGF treatment.

20. The method according to claim 1, wherein the fully human monoclonal antibody ABX-EGF, or antigen binding fragment thereof, exhibits substantially stable pharmacokinetics when administered to the patient.

21. The method according to claim 20, wherein the therapeutically effective amount of the fully human antibody ABX-EGF, or antigen binding fragment thereof, administered to the patient is 0.5 mg/kg to 5 mg/kg.

22. The method according to claim 20, wherein the therapeutically effective amount of the fully human antibody ABX-EGF, or antigen binding fragment thereof, administered to the patient is 1 mg/kg to 2.5 mg/kg.

23. The method according to claim 1, further comprising setting the therapeutically effect amount of the fully human monoclonal antibody ABX-EGF at an amount which triggers a skin rash in the patient.

24. The method of claim 23, wherein the amount which triggers a skin rash in the patient comprises multiple dosages of the ABX-EGF antibody.

25. A kit for treatment of renal carcinoma in a human patient comprising:
a fully human monoclonal antibody ABX-EGF, or fragment thereof, that binds to the epidermal growth factor receptor (EGFr) in a pharmaceutically acceptable carrier; and
instructions for administering to the human patient a therapeutically effective dose of said fully human antibody.

26. The kit of claim 25, wherein the fully human monoclonal antibody ABX-EGF, or fragment thereof, is divided into dosages ranging from 1 mg/kg to 2.5 mg/kg.

27. The kit of claim 26, wherein fully human monoclonal antibody ABX-EGF, or fragment thereof, exhibits substantially stable pharmacokinetics when administered to the patient.

28. An article of manufacture comprising a container, a composition contained therein, and a package insert or label indicating that the composition can be used to treat renal carcinoma characterized by cancer cells expressing epidermal growth factor receptor (EGFr), wherein the composition comprises the fully human monoclonal antibody ABX-EGF, or antigen binding fragments thereof.
